(19) United States
(12) Patent Application Publication
(10) Pub. No.: US 2025/0235105 A1
(43) Pub. Date: Jul. 24, 2025

(54) SYSTEM AND METHOD FOR TIME-RESOLVED FORWARD MODEL FOR MAGNETIC RESONANCE ACOUSTIC RADIATION FORCE IMAGING (MR-ARFI)

(71) Applicant: Case Western Reserve University, Cleveland, OH (US)

(72) Inventors: William A. Grissom, Shaker Heights, OH (US); Kristen Zarcone, Cleveland Heights, OH (US)

(21) Appl. No.: 19/032,819

(22) Filed: Jan. 21, 2025

Related U.S. Application Data
(60) Provisional application No. 63/552,508, filed on Feb. 12, 2024.
(60) Provisional application No. 63/623,686, filed on Jan. 22, 2024.

(51) Int. Cl. A61B 5/00; A61B 5/055

(52) U.S. Cl. CPC A61B 5/0036; A61B 5/0042; A61B 5/0053; A61B 5/055; A61B 2576/026

(57) ABSTRACT

Systems and methods are provided for creating a magnetic resonance acoustic radiation force imaging (MR-ARFI) image from simulations or measurements of a pressure field of an ultrasound transducer. The method includes converting simulations or measurements of a pressure field for an ultrasound transducer to force, delivering the force to a finite element model to calculate dynamic tissue displacements in tissue, delivering the dynamic tissue displacement to control operation of an MR-ARFI process.

### CROSS-REFERENCE TO RELATED APPLICATIONS

This application claims priority to United States Provisional Patent Application Nos. 63/623,686 filed on Jan. 22, 2024, and 63/552,508, filed on Feb. 12, 2024, the entire contents of which are incorporated herein by reference.

### STATEMENT REGARDING FEDERALLY SPONSORED RESEARCH

This invention was made with government support under EB007509 and NS135551 awarded by the National Institutes of Health. The government has certain rights in the invention.

### BACKGROUND

Non-invasive brain stimulation (NIBS) is a well-established approach to study and modulate brain function. Traditional NIBS methods include transcranial electric stimulation (TES) and transcranial magnetic stimulation (TMS) modalities that stimulate the brain by triggering an action potential in neurons through eletrochemical interactions. However, it is challenging to target the deep focal regions of the brain using these methods, because they produce a diffused stimulation field.

Recently, transcranial ultrasound stimulation (TUS) and/or focused ultrasound (FUS) has gained traction as a tool for clinical neuromodulation. Often, TUS and FUS are used somewhat interchangeably, as TUS is a special case of FUS that is configured to generate neuromodulation/neurostimulation. Functionally, the acoustic radiation force (ARF) applied by FUS causes neuromodulation in the brain, and the level and type of neuromodulatory effect changes depending on the pressure of the sound wave. Magnetic resonance acoustic radiation force imaging (MR-ARFI) is an imaging technique that synchronizes high intensity focused ultrasound (HI-FUS) pulses with MR imaging gradient pulses, to encode in the MR image tissue displacement caused by the transference of momentum from the ultrasound pulse to the tissue. Thus, MR-ARFI is used to image displacement due to the ARF in order to localize the FUS beam.

In practice, MR-ARFI synchronizes one or more magnetic field gradient pulses, which are ideally aligned with the focused ultrasound propagation direction at each image location. This creates a phase shift at each spatial location in the image that depends on the tissue location, and tissue displaced by the ultrasound pulse will accrue additional phase because it will be moved to a different point along the magnetic field gradient. If a second measurement is made with the same gradient pulse but without an ultrasound pulse or if the gradient pulse is negated, the difference between the phase of the two measurements will be proportional to the tissue displacement. MR-ARFI is sensitive to tissue displacements as small as 1 micron in vivo.

Despite this sensitivity, accurate control with the TUS or FUS transducer can be difficult. In particular, transmitting sound through intact skull attenuates and shifts the ultrasound focus in ways that depend on both the subject and the transducer position, making it difficult or impossible to know the exact location and amount of tissue affected by TUS/FUS. While MR-ARFI assists with this, there are further complicating factors. For example, current systems are not fully resilient to respiration and gradient eddy currents that can obscure the focus in MR-ARFI maps, which undermines the ability to inherently register anatomic imaging to facilitate targeting specific brain regions. Also, motion from respiration or other physiological processes, such as vascular pulsatility, must be addressed. These challenges are compounded with the need to produce larger displacements with highly focused transducers or the need to relate tissue displacement back to acoustic intensity which determines the degree of neuromodulation.

Thus, there is a continuing need for systems and methods to improve control and management of the delivery of FUS or TUS.

### SUMMARY OF THE DISCLOSURE

The present disclosure addresses the aforementioned drawbacks by providing systems and methods for estimating tissue mechanical properties, acoustic properties, and FUS/TUS transducer properties using MR-ARF data. The properties can then be used to model tissue displacements and improve MR-ARFI processes and/or determine measures for an MRI-ARFI process, even in the face of aberrations or imperfections in the patient's tissue.

In accordance with one aspect of the present disclosure, a method is provided for creating a magnetic resonance acoustic radiation force imaging (MR-ARFI) image from simulations or measurements of a pressure field of an ultrasound transducer. The method includes converting simulations or measurements of a pressure field for an ultrasound transducer to force, delivering the force to a finite element model to calculate dynamic tissue displacements in tissue, delivering the dynamic tissue displacement to control operation of an MR-ARFI process.

In accordance with another aspect of the present disclosure, a non-transitory computer storage medium is provided having instructions stored thereon that, when executed by a processor, cause the processor to carry out steps. The steps can include accessing parameters of ultrasound transducer configured to generate a range of pressure levels, calculating displacements during MR-ARFI pulses based on the generated pressure levels, and determining unknown intensities based on the displacements.

In accordance with yet another aspect of the present disclosure, a method is provided that includes accessing a range of pressure levels generated or capable of being generated in a subject using an ultrasound transducer during a transcranial ultrasound process (TUS) or a focused ultrasound process (FUS). The method also includes, using the computer processor, processing the pressure levels with a model that calculates displacements during a magnetic resonance acoustic radiation force imaging (MR-ARFI) process and interpolating unknown intensities using the displacements. The steps also include, using the computer processor, communicating the displacement to a system performing one of a the TUS or FUS process to facilitate control of the TUS process or FUS process.

In accordance with still another aspect of the present disclosure, a system is provided for estimating tissue mechanical or acoustic properties of a subject using magnetic resonance acoustic radiation force images (MR-ARFI). The system includes a processor configured to access a model of a focused ultrasound transducer and a tissue medium and control the focused ultrasound transducer to acquire MR-ARFI image data from an intended target through the aberrations or imperfections. The processor is also configured to calculate pressure fields generated by the focused ultrasound transducer in a tissue medium of the subject, calculate tissue displacements using the pressure fields using a model, and generate an MR-ARFI image using the tissue displacements and MR-ARFI image data acquired from the subject. The processor is further configured to, using the MR-ARFI image, backpropagate derivatives with respect to parameters of the focused ultrasound transducer or the tissue medium back from the inputs to the model. Also, the processor is configured to, using the derivatives, select updated parameters of the focused ultrasound transducer or the tissue medium that fit the MR-ARFI image data. Further, the processor is configured to, using the updated parameters, select corrective measures that refocuses an ultrasound beam from the focused ultrasound transducer to the intended target through the aberrations or imperfections.

In accordance with another aspect of the disclosure, a method is provided for estimating tissue mechanical or acoustic properties of a subject using magnetic resonance acoustic radiation force images (MR-ARFI). The method includes accessing a model of a focused ultrasound transducer or a tissue medium, controlling the focused ultrasound transducer to acquire MR-ARFI image data from an intended target through the aberrations or imperfections, and, using a differentiable acoustic solver, calculating pressure fields generated by the focused ultrasound transducer in a tissue medium of the subject. The method also includes, using a finite element method (FEM) solver, calculating tissue displacements using the pressure fields and generating an MR-ARFI image using the tissue displacements and MR-ARFI image data acquired from the subject. The method further includes, using the MR-ARFI image, backpropagating derivatives with respect to parameters of the focused ultrasound transducer or the tissue medium back from the inputs to the FEM solver. Furter, the method includes using the derivatives, selecting updated parameters of the focused ultrasound transducer or the tissue medium that fit the MR-ARFI image data, and, using the updated parameters, selecting corrective measures that refocuses an ultrasound beam from the focused ultrasound transducer to the intended target through the aberrations or imperfections.

These are but a few, non-limiting examples of aspects of the present disclosures. Other features, aspects and implementation details will be described hereinafter.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 is a block diagram of an example MR-ARFI system configured for Mag-ARFI in accordance with the present disclosure.

FIG. 2 is a further block diagram of an example MR-ARFI system of FIG. 1 in accordance with the present disclosure.

FIG. 3A is a graphic illustration of a process for creating displacement maps in accordance with the present disclosure.

FIG. 3B is a flow chart setting forth one, non-limiting example of a process for determining dynamic tissue displacements and controlling an MR-ARFI process in accordance with the present disclosure.

FIG. 4 is a flow chart setting forth one, non-limiting example of a process for determining corrective or compensation measures for controlling an MR-ARFI process in accordance with the present disclosure.

### DETAILED DESCRIPTION

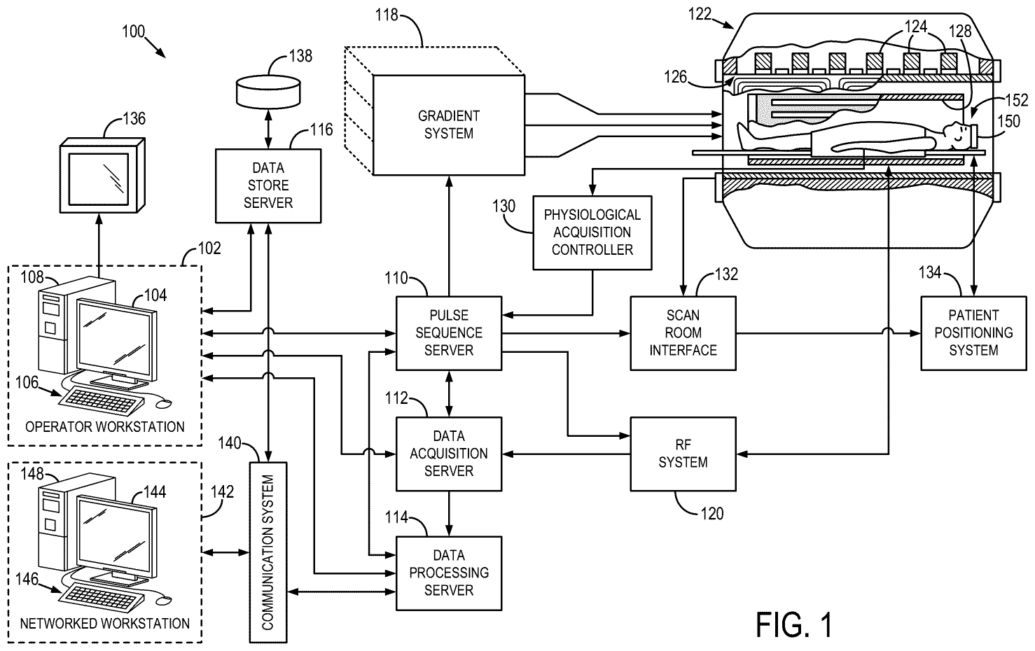

Referring to FIG. 1, an example of an MRI system 100 configured for operation in accordance with the present disclosure. The MRI system 100 includes an operator workstation 102 that may include a display 104, one or more input devices 106 (e.g., a keyboard, a mouse), and a computer or processor 108. The processor 108 may include a commercially available programmable machine running a commercially available operating system. The operator workstation 102 provides an operator interface that facilitates entering scan parameters into the MRI system 100. The operator workstation 102 may be coupled to different servers, including, for example, a pulse sequence server 110, a data acquisition server 112, a data processing server 114, and a data store server 116. The MRI system 100 also includes gradient system 118 and a radio frequency (RF) system 120. A magnet assembly 122 includes a polarizing magnet 124, which may be a low-field magnet. The gradient system 118 may control a gradient coil assembly 126. The MRI system 100 may optionally include a whole-body RF coil 128, or a local coil system (not shown) that is controlled by the RF system 120.

The pulse sequence server 110 functions in response to instructions provided by the operator workstation 102 to operate a gradient system 118 and the RF system 120. Gradient waveforms for performing a prescribed scan are produced and applied to the gradient system 118, which then excited gradient coils in the assembly 126 to produce the magnetic field gradients (e.g., Gₓ, G_{y}, and G_{z}) that can be used for spatially encoding magnetic resonance signals.

RF waveforms are applied by the RF system 120 to the RF coil 128, or a separate local coil to perform the prescribed magnetic resonance pulse sequence. Responsive magnetic resonance signals detected by the RF coil 128, or a separate local coil, are received by the RF system 120. The responsive magnetic resonance signals may be amplified, demodulated, filtered, and digitized under direction of commands produced by the pulse sequence server 110. The RF system 120 includes an RF transmitter for producing a wide variety of RF pulses used in MRI pulse sequences. The RF transmitter is responsive to the prescribed scan and direction from the pulse sequence server 110 to produce RF pulses of the desired frequency, phase, and pulse amplitude waveform. The generated RF pulses may be applied to the whole-body RF coil 928 or to one or more local coils or coil arrays.

The RF system 120 also includes one or more RF receiver channels. An RF receiver channel includes an RF preamplifier that amplifies the magnetic resonance signal received by the coil 128 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{( {I}^{2} + {Q}^{2} )}$$

and the phase of the received magnetic resonance signal may also be determined according to the following relationship:

$$ϕ = {tan}^{- 1} ( \frac{Q}{I} )$$

The pulse sequence server 110 may receive patient data from a physiological acquisition controller 130. Byway of example, the physiological acquisition controller 130 may receive signals from a number of different sensors connected to the patient, including electrocardiograph (ECG) signals from electrodes, or respiratory signals from a respiratory bellows or other respiratory monitoring devices. These signals may be used by the pulse sequence server 910 to synchronize, or “gate,” the performance of the scan with the subject's heartbeat or respiration.

The pulse sequence server 110 may also connect to a scan room interface circuit 132 that receives signals from various sensors associated with the condition of the patient and the magnet system. Through the scan room interface circuit 132, a patient positioning system 134 can receive commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage, so that data are not lost by data overrun. In some scans, the data acquisition server 112 passes the acquired magnetic resonance data to the data processor server 114. In scans that require information derived from acquired magnetic resonance data to control the further performance of the scan, the data acquisition server 112 may be programmed to produce such information and convey it to the pulse sequence server 110. For example, during pre-scans, magnetic resonance data may be acquired and used to calibrate the pulse sequence performed by the pulse sequence server 110. As another example, navigator signals may be acquired and used to adjust the operating parameters of the RF system 120 or the gradient system 118, or to control the view order in which k-space is sampled. In still another example, the data acquisition server 112 may also process magnetic resonance signals used to detect the arrival of a contrast agent in a magnetic resonance angiography (MRA) scan. For example, the data acquisition server 112 may acquire magnetic resonance data and processes it in real-time to produce information that is used to control the scan.

The data processing server 114 receives magnetic resonance data from the data acquisition server 112 and processes the magnetic resonance data in accordance with instructions provided by the operator workstation 102. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 114 are conveyed back to the operator workstation 102 for storage. Real-time images may be stored in a data base memory cache, from which they may be output to operator display 102 or a display 136. Batch mode images or selected real time images may be stored in a host database on disc storage 138. When such images have been reconstructed and transferred to storage, the data processing server 114 may notify the data store server 116 on the operator workstation 102. The operator workstation 102 may be used by an operator to archive the images, produce films, or send the images via a network to other facilities.

The MRI system 100 may connect through a communication system 140 to one or more networked workstations 142. For example, a networked workstation 142 may include a display 144, one or more input devices 146 (e.g., a keyboard, a mouse), and a computer or processor 148. The networked workstation 142 may be located within the same facility as the operator workstation 102, or in a different facility, such as a different healthcare institution or clinic, and be configured for communication therebetween. Also, the networked workstation 142 may form a server or represent or be connected to any remote communication devices, including portable electronics or cloud servers. Additionally, regardless of the particular hardware or implementation, the networked workstation 142 may gain remote access to the data processing server 114 or data store server 116 via the communication system 140. Also, multiple networked workstations 142 may have access to the data processing server 114 and the data store server 116. In this manner, magnetic resonance data, reconstructed images, or other data may be exchanged between the data processing server 114 or the data store server 116 and the networked workstations 142, such that the data or images may be remotely processed by a networked workstation 142.

As will be described, the above-described 100 system may be configured as a MR-ARFI or magnitude-contrast MR-acoustic radiation force imaging (Mag-ARFI) system. As such, the system 100 may include a transducer system 150 coupled to a patient 152 when arranged for MR imaging.

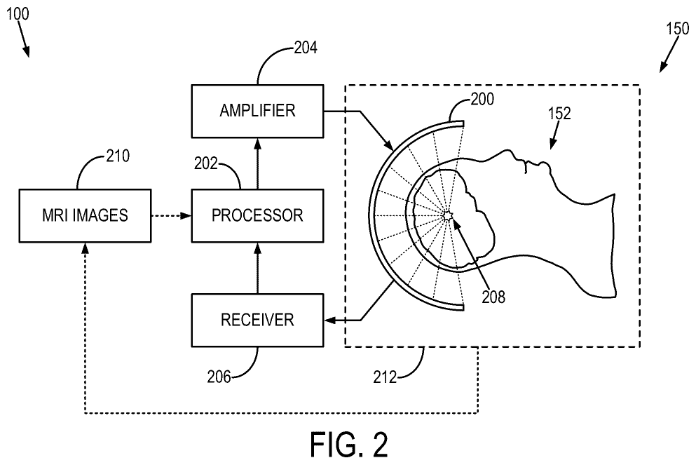

Referring now to FIG. 2, the transducer system 150 may be configured more particularly for transcranial ultrasound stimulation (TUS) of the patient 152. That is, the subject 152 receives ultrasound energy from one or a plurality of transducer elements 200. In one non-limiting configuration, if an array of transducers is utilized, the transducer elements 200 may be arranged in a matrix that surrounds an extent of the patient's head. For example, the transducer system 150 may be a hemispherical array of transducer elements 200. The transducer system 150 may include a processor 202 that is in communication with an amplifier 204 and a receiver 206. The amplifier 204 may receive driving signals from the processor 202 and, in turn, direct the transducer elements of the transducer system 150 to generate ultrasound energy. The receiver 206 may receive acoustic signals during sonications and relay these signals to the processor 202 for processing in accordance with the present disclosure. The processor 202 may also be configured to adjust the driving signals in response to the acoustic signals received by the receiver 206. For example, the phase and/or amplitude of the driving signals may be adjusted so that ultrasound energy is more efficiently transmitted through the skull of the subject 152 and into the target volume-of-interest 208. Furthermore, the acoustic signals may also be analyzed to determine whether and how the extent of the focal region should be adjusted. As will be described below in detail, MRI images 210 may also be used to guide the application of ultrasound energy to the subject 152. Thus, an MRI system described with respect to FIG. 1, generally indicated as dashed box 212, may be used to acquired MRI images 210 of the subject 152. The MRI images 210 may then be provided to the processor 202 to adjust the parameters of the sonications. For example, the phase and/or amplitude of the driving signals may be adjusted so that ultrasound energy is more efficiently transmitted through the skull of the subject 152 and into the target volume-of-interest 208.

MRI-acoustic radiation force imaging is an imaging technique that synchronizes high intensity ultrasound pulses with MR imaging gradient pulses, to encode in the MR image tissue displacement caused by the transference of momentum from the ultrasound pulse to the tissue. It is currently used to target focused ultrasound in the brain, but the actual amplitude of the measurements is discarded because it is not yet known how to relate the displacement back to ultrasound measures that dictate tissue response such as pressure, intensity, or mechanical index.

The present disclosure provides systems and methods for using the quantitative tissue displacement measurement provided by MR-ARFI to relate back to acoustic intensity via a comprehensive forward model relating the FUS beam to the MR-ARFI image. Thus, the present disclosures provides systems and methods for using a forward model relating pressure to MR-ARFI displacement. That is, a system and method are provided to predict the amount of displacement at steady state has been proposed based on the use of ultrasound pulses long enough (up to several ms long) to reach steady-state. It is desirable to use short ARF pulses in MR-ARFI to minimize bioeffects, which can break the steady state assumption and lead to errors. That is, the present disclosure recognizes that the displacement amplitudes have the potential to provide acoustic dosimetry needed to predict neuromodulatory effects, if they can be related back to acoustic intensity or pressure. Thus, a robust forward model is provided to calculate the expected displacement map measured by MR-ARFI, which can enable recovery of acoustic intensity from imaged displacements. This can be done, for example, using a lookup table method or AI model. This enables the use of MR-ARFI for dosimetry in FUS/TUS neuromodulation.

MR-ARFI is an imaging method that reports the average tissue displacement in each voxel of an image that is caused by a focused ultrasound pulse played during a motion encoding gradient pulse. However, the present disclosure seeks to acquire multiple MR-ARFI measurements with different pulse durations with the goal of weighting the signal more towards the plateaus or rises and falls of the displacement, so that both the tissue's viscoelastic properties (e.g., under a Kelvin-Voigt model), and the acoustic intensity at each spatial location can be simultaneously estimated.

Let us assume that the displacement is a separable function of time and vector-valued space, where the spatial function is \vec{u}(\vec{x}) and the temporal component is a decaying exponential rise during the ARF pulse. Alternatively, the displacement can be expressed as a weighted sum of such functions, for example, with different time constants τ and different spatial components \vec{u}(\vec{x}). In the case of a single separable function, we have a during-ARF spatiotemporal displacement function of:

$$\vec{U} ( \vec{x} , t ) = ( 1 - {e}^{- t / τ} ) \vec{u} ( \vec{x} ) . \qquad ( 1 )$$

We further assume that after the ARF pulse the displacement also decays with the same time constant, leading to a post-ARF spatiotemporal displacement of:

$$\vec{U} ( \vec{x} , t ) = ( 1 - {e}^{- T / τ} ) {e}^{- t / τ} \vec{u} ( \vec{x} ) . \qquad ( 2 )$$

In these expressions, τ=η/λ is the unknown tissue time constant equal to the ratio of the unknown viscosity (λ; Pa s) and elasticity (λ; Pa), and T is the ARF pulse duration. The vector-valued displacement field will u(\vec{x}) be represented as a sum of weighted Gaussian functions, for example for the x-component:

$${u}_{x} ( x ) = {\sum }_{i = 1}^{{N}_{g}} {α}_{i}^{x} exp ( - \frac{1}{2} ( x - {μ}_{i} ) {\sum }_{i}^{- 1} ( x - {μ}_{i} ) ) ; \qquad ( 3 )$$

where x=[x y z] is a spatial coordinate, N_{g} is the number of Gaussian functions, x is the coefficient of the i^{th} Gaussian, μᵢ is the center of the i^{th} Gaussian, and Σᵢ is the covariance matrix of the i^{th} Gaussian. This radial basis function approach could be realized using any parameterizable basis function that is differentiable, such as, a Lorentzian or a ‘bump’ function. Though we do not implement it here, μᵢ and Σᵢ could be different for the x, y, and z vector components. In this work we set the μᵢ's and Σᵢ's a priori and hold them fixed, and solve for the a coefficients.

MR-ARFI provides measurements of average displacement during a motion-encoding gradient (MEG) along one spatial dimension at a time, which is usually chosen to coincide with the ultrasound axial dimension. Here we will denote that dimension as z. In the absence of other physical constraints on the displacement field model, the z-component of the model can be fit to MR-ARFI measurements by minimizing the sum-of-squared errors between the model and the measured displacement maps:

$${\sum }_{i = 1}^{{N}_{g}} {\vert d ( {\vec{x}}_{i} ) - {\int }_{MEG} {U}_{z} ( {\vec{x}}_{i} , t ) dt \vert}^{2} , \qquad ( 4 )$$

where Nₓ is the number of spatial locations from the displacement map that are used in the fitting, and the time integral is calculated over the motion-encoding gradient (MEG) duration. This function is convex-quadratic in the collected vector of displacement z-coefficients α_{z} and can be solved for α_{z} by forming a matrix of the Gaussian functions with unit coefficients that are sampled on the same spatial grid as the measurements, and applying pseudoinverse. One also can also determine the time constant τ which will be discussed below.

To solve for the force, we can simultaneously require the fitted vector-valued displacements to match the data, and to satisfy the Cauchy momentum equation:

$$ρ \frac{{d}^{2} \vec{U}}{{dt}^{2}} = ∇ ▯ \overline{\overline{σ}} + \vec{b} , \qquad ( 5 )$$

where ρ is the tissue density, \vec{b} is the acoustic radiation force, and [symbol] is the divergence of the Cauchy stress tensor. We have suppressed spatiotemporal dependencies for brevity. The Kelvin-Voigt model is commonly used for ruibbery materials and tissues that resist sudden deformations. Under the model, the Cauchy stress tensor is given by:

$$\overline{\overline{σ}} = λ tr ( \overline{\overline{ò}} ) I + η \frac{d \overline{\overline{ò}}}{dt} , \qquad ( 6 )$$

where λ is the material's elasticity in Pascal and η is the material's viscosity in Pa·s. The time constant τ in our model is equal to the ratio of η and λ:

$$τ = \frac{η}{λ} , \qquad ( 7 )$$

where o̅ in (6) is the isotropic strain tensor, which is calculated from the gradient of the vector displacement field as:

$$\overline{\overline{ò}} = ∇ \vec{U} + {( ∇ \vec{U} )}^{T} , \qquad ( 8 )$$

where ^{T} denotes matrix transpose. Substituting the Kelvin-Voigt stress tensor into the Cauchy momentum equation, we obtain:

$$ρ \frac{{d}^{2} \vec{U}}{{dt}^{2}} = ∇ ▯ ( λ tr ( \overline{\overline{ò}} ) I + η \frac{d \overline{\overline{ò}}}{dt} ) + \vec{b} . \qquad ( 9 )$$

This equation involves first- and second-order temporal derivatives and second-order spatial derivatives of the vector displacement field, but these are trivial to compute analytically for our displacement model. If we substitute our model into (9) and assume we have already applied the spatial derivatives to the vector displacement field yielding a spatial elastic term \vec{e}(\vec{x};α) and a spatial viscous term \vec{v}(\vec{x};α), we obtain during the pulse:

$$ρ \frac{{d}^{2}}{{dt}^{2}} ( 1 - {e}^{- \frac{t}{τ}} ) \vec{u} ( \vec{x} ; α ) = λ ( 1 - {e}^{- t / τ} ) \vec{e} ( \vec{x} ; α ) + η \frac{d}{dt} ( 1 - {e}^{- t / τ} ) \vec{v} ( \vec{x} ; α ) + \vec{b} ( \vec{x} ) . \qquad ( 10 )$$

Applying the time derivatives then yields:

$$- ρ \frac{{e}^{- t / τ}}{{τ}^{2}} \vec{u} ( \vec{x} ; α ) = λ ( 1 - {e}^{- t / τ} ) \vec{e} ( \vec{x} ; α ) + η \frac{{e}^{- t / τ}}{τ} \vec{v} ( \vec{x} ; α ) + \vec{b} ( \vec{x} ) . \qquad ( 11 )$$

This realization and framework allows for a solution for the vector displacement field and, subsequently, the acoustic radiation force. That is, for the above equation to be satisfied for all time, the sums of time-dependent terms on each side of the equation must be equal:

$$- \frac{ρ}{{τ}^{2}} \vec{u} ( \vec{x} ; α ) = - λ \vec{e} ( \vec{x} ; α ) + \frac{η}{τ} \vec{v} ( \vec{x} ; α ) ; \qquad ( 12 )$$

$$= - λ \vec{e} ( \vec{x} ; α ) + λ \vec{v} ( \vec{x} ; α ) ; \qquad ( 13 )$$

and likewise, the sums of the time-independent terms on each side must be:

$$0 = λ \vec{e} ( \vec{x} ; α ) + \vec{b} ( \vec{x} ) . \qquad ( 14 )$$

Then, the vector-valued displacement field parameters (principally, the vector of weights α) can be recovered. For example, the vector-valued displacement field parameters can be recovered by simultaneously requiring the field to match the data. In one non-limiting example, this can be implemented by minimizing (4), and to satisfy (13). In this case, α is available and (14) can be used to calculate a desired acoustic radiation force vector \vec{b}(\vec{x}) from the elastic term.

Because \vec{u}(\vec{x}; α), \vec{e}(\vec{x}; α), and \vec{v}(\vec{x}; α) are linear functions of α, (13) and (14) can be discretized and rewritten in matrix-vector form:

$$( - \frac{ρ}{{τ}^{2}} U + λ E - λV ) α = 0 ; \qquad ( 15 )$$

$$- λ E α = b ; \qquad ( 16 )$$

where U relates the Gaussian coefficients to the vector dispalcement field, E relates them to the vector-valued elastic term, and V relates them to the vector-valued viscous term. In this, b is the discretized acoustic radiation force vector field. The discretized x, y, and z components of the vector fields are stacked in the row dimension of the matrices and in b. The “Cauchy” matrix can then be defined as:

$$C \hat{=} - \frac{ρ}{{τ}^{2}} U + λ E - λ V ; \qquad ( 17 )$$

which shows that the coefficient vector α lies in the null space of C (i.e. Cα=0), such that a satisfies the Cauchy equation. Thus, under this displacement model and the Kelvin-Voigt model, the overall problem of solving for the displacement vector field that both matches the measured displacement maps and satisfies the Cauchy momentum equation can be stated as a null-space problem of the form:

$$minimize {‖ d - {U}_{d} α ‖}^{2} \qquad ( 18 )$$

$$subject to C α = 0 ;$$

where d collects all the measured displacement maps, and U_{d} is a matrix relating the coefficients in α to the sampled displacement field, with the same vector directions as d.

For fixed λ, η, and τ=η/λ, the optimization problem in (18) is convex and can be solved using a least-square solver, where the null-space constraint is implemented using regularization, effectively solving:

$$minimize {‖ d - {U}_{d} α ‖}^{2} + ℽ {‖ C α ‖}^{2} . \qquad ( 19 )$$

where γ is a user-selected regularization parameter that balances the two terms. Alternatively, if a reduced basis B for the null-space of C can be calculated (e.g., form the right singular vectors of C), then the problem can be solved using this basis as:

$$minimize {‖ d - {U}_{d} B {α}^{r} ‖}^{2} ; \qquad ( 20 )$$

and the full set of coefficients can be recovered from α=Bα^{r}.

In practice, there is not perfect knowledge of λ and η upon initialization, so λ and η can be be jointly solved for using α. To achieve this, starting with initial values for λ and η, as described above, updates to α can be alternated with updates to λ and η, holding α fixed. Since λ and η are positive-valued scalar parameters for which reasonable ranges of feasible values can be defined, they can be efficiently updated by holding a fixed, pre-calculating all the matrix-vector products in (18), and applying a grid search.

An alternative strategy is to first fit the coefficients of the measured components of α (the z-component in a typical MR-ARFI dataset), along with τ, to the data. Then, the remaining vector components and η, λ can be fit to satisfy the Cauchy equation, holding τ fixed. Finally, the force can be calculated using (16).

The above-described mathematical constructs approach the matter as a 3D matrix-vector problem. However, this is not the only mathematical approach or construct that can be used within the context of the present disclosure.

It is possible that more than one exponential will be required to accurately model the displacement timecourse. In this case, the displacement would be modeled as:

$$\vec{U} ( \vec{x} , t ) = {\sum }_{i = 1}^{{N}_{e}} ( 1 - {e}^{- \frac{t}{{τ}_{i}}} ) \vec{{u}_{l}} ( \vec{x} ; {α}_{i} ) ; \qquad ( 21 )$$

during the pulse, and

$$\vec{U} ( \vec{x} , t ) = {\sum }_{i = 1}^{{N}_{e}} ( 1 - {e}^{- \frac{T}{{τ}_{i}}} ) {e}^{- \frac{t}{{τ}_{i}}} \vec{{u}_{l}} ( \vec{x} ; {α}_{i} ) ; \qquad ( 22 )$$

after the pulse. Each exponential could also have an independent time delay. Under this model, the displacement is modeled as a sum of separable spatiotemporal functions, each with its own time constant and spatial vector displacement field. Since the Cauchy equation is a linear function of displacement and the exponentials are linearly independent, the sums of time-dependent terms must be equal on either side of the equation for each time constant. This yields a set of matrix-vector equations:

$$( - \frac{ρ}{{τ}_{1}^{2}} U + {λ}_{1} E - {λ}_{1} V ) {α}_{1} = 0 ; \qquad ( 23 )$$

$$…$$

$$( - \frac{ρ}{{τ}_{{N}_{e}}^{2}} U + {λ}_{{N}_{e}} E - {λ}_{{N}_{e}} V ) {α}_{{N}_{e}} = 0 ; \qquad ( 24 )$$

$${\sum }_{i = 1}^{{N}_{e}} {λ}_{1} E {α}_{i} = b . \qquad ( 25 )$$

Either fitting strategy could be adapted to solve these equations and obtain b. For example, the alternative strategy described at the end of the previous section would proceed by fitting the combined displacement vector field model to the measured displacements to obtain the τᵢ's and the a_{z,i}'s, then individually solving for the remaining vector components and the λᵢ's by solving each of the above (23) and (24). Finally, (25) would be evaluated to obtain the force vector b.

Turning back to (5)-(8), the expression

$$ϵ = \frac{1}{2} ( ∇ u + {( ∇ u )}^{T} )$$

can be expanded as:

$$ϵ = ( \begin{matrix}{ϵ}_{xx} & {ϵ}_{xy} & {ϵ}_{xz} \\ {ϵ}_{yx} & {ϵ}_{yy} & {ϵ}_{yz} \\ {ϵ}_{zx} & {ϵ}_{zy} & {ϵ}_{zz}\end{matrix} ) ; \qquad ( 26 )$$

$$= ( \begin{matrix}\frac{\partial {u}_{x}}{{\partial }_{x}} & \frac{1}{2} ( \frac{\partial {u}_{x}}{{\partial }_{y}} + \frac{\partial {u}_{y}}{{\partial }_{x}} ) & \frac{1}{2} ( \frac{\partial {u}_{x}}{{\partial }_{z}} + \frac{\partial {u}_{z}}{{\partial }_{x}} ) \\ \frac{1}{2} ( \frac{\partial {u}_{y}}{{\partial }_{x}} + \frac{\partial {u}_{x}}{{\partial }_{y}} ) & \frac{\partial {u}_{y}}{{\partial }_{y}} & \frac{1}{2} ( \frac{\partial {u}_{y}}{{\partial }_{z}} + \frac{\partial {u}_{z}}{{\partial }_{y}} ) \\ \frac{1}{2} ( \frac{\partial {u}_{z}}{{\partial }_{x}} + \frac{\partial {u}_{x}}{{\partial }_{z}} ) & \frac{1}{2} ( \frac{\partial {u}_{z}}{{\partial }_{y}} + \frac{\partial {u}_{y}}{{\partial }_{z}} ) & \frac{\partial {u}_{z}}{{\partial }_{z}}\end{matrix} ) . \qquad ( 27 )$$

Strain further involves the trace of ∈, which is

$$tr ( ϵ ) = \frac{\partial {u}_{x}}{{\partial }_{x}} + \frac{\partial {u}_{y}}{{\partial }_{y}} + \frac{\partial {u}_{z}}{{\partial }_{z}} .$$

Let's apply the divergence operator to the elastic and viscosity terms separately. The divergence of a tensor is the vector whose components are the divergences of the rows or columns of the tensor, depending on the convention used. For a symmetric tensor like ∈, either convention works. So the divergence of the elastic term (assuming λ is constant over space) is:

$$λ ∇ \cdot ( tr ( ϵ ) I ) = λ ( \begin{matrix}\frac{{\partial }^{2} {u}_{x}}{\partial {x}^{2}} + \frac{{\partial }^{2} {u}_{y}}{\partial x \partial y} + \frac{{\partial }^{2} {u}_{z}}{\partial x \partial z} \\ \frac{{\partial }^{2} {u}_{x}}{\partial y \partial x} + \frac{{\partial }^{2} {u}_{y}}{\partial {y}^{2}} + \frac{{\partial }^{2} {u}_{z}}{\partial y \partial z} \\ \frac{{\partial }^{2} {u}_{x}}{\partial z \partial x} + \frac{{\partial }^{2} {u}_{y}}{\partial z \partial y} + \frac{{\partial }^{2} {u}_{z}}{\partial {z}^{2}}\end{matrix} ) ; \qquad ( 28 )$$

and the divergence of the viscosity term (assuming η is constant over space and that u and subsequently ε are separable functions of space and time) is:

$$η \frac{\partial }{\partial t} ∇ \cdot ϵ = η \frac{\partial }{\partial t} ( \begin{matrix}\frac{{\partial }^{2} {u}_{x}}{\partial {x}^{2}} + \frac{1}{2} ( \frac{{\partial }^{2} {u}_{x}}{\partial {y}^{2}} + \frac{{\partial }^{2} {u}_{y}}{\partial y \partial x} ) + \frac{1}{2} ( \frac{{\partial }^{2} {u}_{x}}{\partial {z}^{2}} + \frac{{\partial }^{2} {u}_{z}}{\partial z \partial x} ) \\ \frac{1}{2} ( \frac{{\partial }^{2} {u}_{y}}{\partial {x}^{2}} + \frac{{\partial }^{2} {u}_{x}}{\partial x \partial y} ) + \frac{{\partial }^{2} {u}_{y}}{\partial {y}^{2}} + \frac{1}{2} ( \frac{{\partial }^{2} {u}_{y}}{\partial {z}^{2}} + \frac{{\partial }^{2} {u}_{z}}{\partial z \partial y} ) \\ \frac{1}{2} ( \frac{{\partial }^{2} {u}_{z}}{\partial {x}^{2}} + \frac{{\partial }^{2} {u}_{x}}{\partial x \partial z} ) + \frac{1}{2} ( \frac{{\partial }^{2} {u}_{z}}{\partial {y}^{2}} + \frac{{\partial }^{2} {u}_{y}}{\partial y \partial z} ) + \frac{{\partial }^{2} {u}_{z}}{\partial {z}^{2}}\end{matrix} ) . \qquad ( 29 )$$

So, if u_{*} (where * is x, y, or z) is expressed as the sum of a set of Gaussian functions, the second spatial derivatives of those Gaussian functions are needed. These are given by:

$$\frac{{\partial }^{2} G ( x )}{\partial {x}_{i} \partial {x}_{j}} = G ( x ) ( {\sum }_{k , m} {\sum }_{ik}^{- 1} {\sum }_{jm}^{- 1} ( {x}_{k} - {μ}_{k} ) ( {x}_{m} - {μ}_{m} ) - {\sum }_{ij}^{- 1} ) . \qquad ( 30 )$$

To test this, a 1D version of the analytical model can be considered. To do so, a force and displacement in x can be assumed and the spatial component of displacement can be defined as U(x), given by:

$$U ( x ) = {\sum }_{i = 1}^{{N}_{g}} {α}_{i} exp ( - \frac{{( x - {μ}_{i} )}^{2}}{2 {σ}_{i}^{2}} ) . \qquad ( 31 )$$

Then the displacement during the pulse is given by:

$$u ( x , t ) = ( 1 - {e}^{- t / τ} ) U ( x ) , \qquad ( 32 )$$

and after the pulse is:

$$u ( x , t ) = ( 1 - {e}^{- T / τ} ) {e}^{- ( t - T ) / τ} U ( x ) . \qquad ( 33 )$$

Using this, the tissue acceleration

$$u ( x , t ) = ( 1 - {e}^{- T / τ} ) {e}^{- ( t - T ) / τ} U ( x ) . \qquad ( 33 )$$

can be defined, which during the pulse, is given by:

$$ρ \frac{{\partial }^{2} u}{\partial {t}^{2}} = - \frac{ρ}{{τ}^{2}} {e}^{- t / τ} U ( x ) ; \qquad ( 34 )$$

And, after the pulse, is given by:

$$ρ \frac{{\partial }^{2} u}{\partial {t}^{2}} = \frac{ρ}{{τ}^{2}} ( 1 - {e}^{- T / τ} ) {e}^{- ( t - T ) / τ} U ( x ) . \qquad ( 35 )$$

During the pulse, the elastic term is given by:

$$λ ∇ \cdot ( tr ( ϵ ) I ) = λ ( 1 - {e}^{- t / τ} ) \frac{{\partial }^{2} U ( x )}{\partial {x}^{2}} ; \qquad ( 36 )$$

$$= λ ( 1 - {e}^{- t / τ} ) \frac{{\partial }^{2}}{\partial {x}^{2}} {\sum }_{i = 1}^{{N}_{g}} {α}_{i} exp ( - \frac{{( x - {μ}_{i} )}^{2}}{2 {σ}_{i}^{2}} ) ; \qquad ( 37 )$$

$$= λ ( 1 - {e}^{- t / τ} ) {\sum }_{i = 1}^{{N}_{g}} {α}_{i} ( \frac{{( x - {μ}_{i} )}^{2}}{{σ}_{i}^{4}} - \frac{1}{{σ}_{i}^{2}} ) exp ( - \frac{{( x - {μ}_{i} )}^{2}}{2 {σ}_{i}^{2}} ) ; \qquad ( 38 )$$

and after the pulse it is given by:

$$( 39 )$$

$$λ ∇ \cdot ( tr ( ϵ ) I ) = λ ( 1 - {e}^{- T / τ} ) {e}^{- ( t - T ) / τ} {\sum }_{i = 1}^{{N}_{g}} {α}_{i} ( \frac{{( x - {μ}_{i} )}^{2}}{{σ}_{i}^{4}} - \frac{1}{{σ}_{i}^{2}} ) exp ( - \frac{{( x - {μ}_{i} )}^{2}}{2 {σ}_{i}^{2}} ) .$$

The viscosity term during the pulse is:

$$η \frac{\partial }{\partial t} ∇ \cdot ϵ = η \frac{{\partial }^{3} u}{\partial t \partial {x}^{2}} ; \qquad ( 40 )$$

$$= \frac{η}{τ} {e}^{- t / τ} {\sum }_{i = 1}^{{N}_{g}} {α}_{i} ( \frac{{( x - {μ}_{i} )}^{2}}{{σ}_{i}^{4}} - \frac{1}{{σ}_{i}^{2}} ) exp ( - \frac{{( x - {μ}_{i} )}^{2}}{2 {σ}_{i}^{2}} ) ; \qquad ( 41 )$$

and after the pulse is:

$$( 42 )$$

$$η \frac{\partial }{\partial t} ∇ \cdot ϵ = - \frac{η}{τ} ( 1 - {e}^{- T / τ} ) {e}^{- ( t - T ) / τ} {\sum }_{i = 1}^{{N}_{g}} {α}_{i} ( \frac{{( x - {μ}_{i} )}^{2}}{{σ}_{i}^{4}} - \frac{1}{{σ}_{i}^{2}} ) exp ( - \frac{{( x - {μ}_{i} )}^{2}}{2 {σ}_{i}^{2}} ) .$$

so the b equation during the pulse is:

$$b ( x ) = - ( λ \frac{{\partial }^{2} u}{\partial {x}^{2}} + η \frac{{\partial }^{3} u}{\partial t \partial {x}^{2}} ) - \frac{ρ}{{τ}^{2}} {e}^{- t / τ} {\sum }_{i = 1}^{{N}_{g}} {α}_{i} G ( x , {μ}_{i} , {σ}_{i} ) ; \qquad ( 43 )$$

and after the pulse is:

$$b ( x ) = - ( λ \frac{{\partial }^{2} u}{\partial {x}^{2}} + η \frac{{\partial }^{3} u}{\partial t \partial {x}^{2}} ) + \frac{ρ}{{τ}^{2}} ( 1 - {e}^{- T / τ} ) {e}^{- ( t - T ) / τ} {\sum }_{i = 1}^{{N}_{g}} {α}_{i} G ( x , {μ}_{i} , {σ}_{i} ) . \qquad ( 44 )$$

This construct can be applied in any direction and, as described above, matrix operations can be used to yield the 3D implementation, if desired.

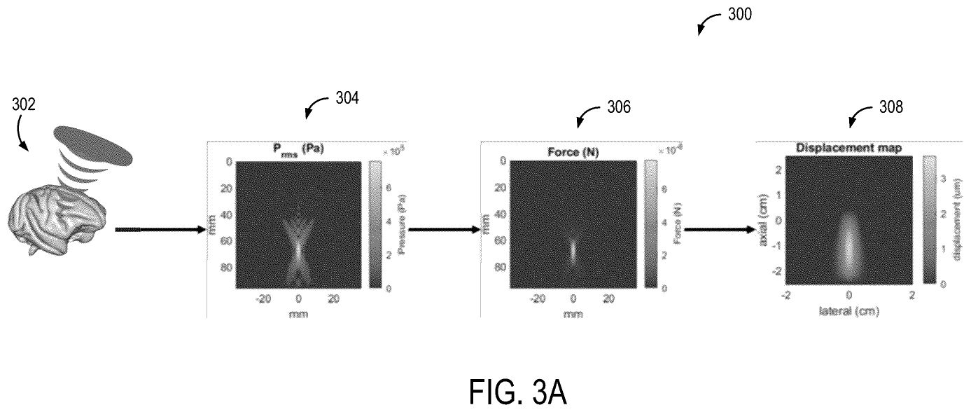

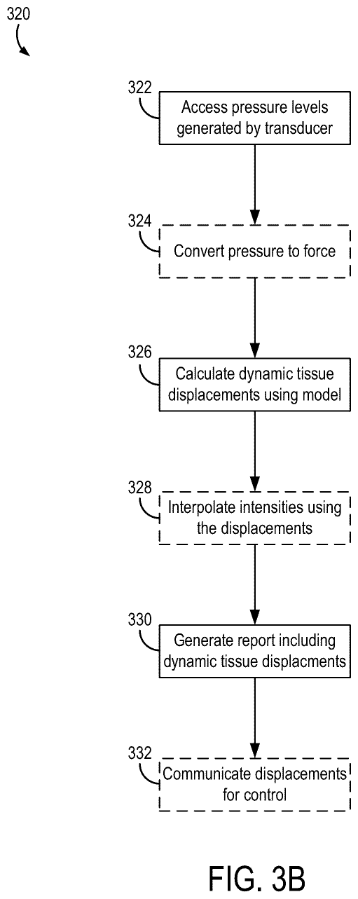

Referring now to FIG. 3A, the above-described process can be used to create an MR-ARFI displacement map and/or model. For example, a process 300 for creating a displacement map forward model can begin with transducer data 302. The transducer data may be acquired from a particular transducer or can include data simulated at a desired free-field pressure level in, for example, k-Wave. In either case, time-resolved pressure maps 304 can be produced that can then be used to calculate acoustic intensity and force 306. The force field can then be input to a finite element method (FEM) solver (as one non-limiting example, LS-DYNA or JAX-FEM may be used) for the duration of the MR-ARFI motion encoding gradient and the time-resolved displacements can be averaged to obtain a predicted displacement map 308. Also, the force is equal to the vector b in (14). Thus, instead of using a FEM solver, this relationship can be used to calculate the resulting displacements under the separable model.

Referring to FIG. 3B, implementation of the above-described concepts in a clinical process 320 may begin at process block 322 with accessing a range of pressure levels generated or capable of being generated in a subject using an ultrasound transducer during a transcranial ultrasound process or a focused ultrasound process. At process block 324, the pressure levels may be converted to force. At process block 326, a model is used to calculate dynamic tissue displacements in tissue during an MR-ARFI process, either from the original pressures or converted force. If needed, the process 320 may include interpolating unknown intensities using the displacements at process block 328. At process block 330, a report may be generated that includes the dynamic tissue displacements determined from the MR-ARFI data. At process block 332, the report may be communicated to deliver the displacement to a system performing the MR-ARFI process and, more particularly, to control one of a transcranial ultrasound stimulation process (TUS) or focused ultrasound (FUS) process performed as part of the MR-ARFI process.

The forward algorithm to calculate tissue displacement over time within a tissue mesh, as described above, can be formulated using known tissue viscoelastic properties and acoustic radiation force. For purposes of illustration only the FEM solver may be JAX-FEM. Implementing this algorithm in a FEM solver then makes this forward process differentiable, so that all parameters can be estimated from measured MR-ARFI images. This alternative to the above matrix/vector approach also relates pressure/intensity/force to the measured displacement.

JAX-FEM can solve static problems of the form:

$$- ∇ \cdot σ = b in Ω ; \qquad ( 45 )$$

$$u = {u}_{D} on {Γ}_{D} ; \qquad ( 46 )$$

$$σ \cdot n = t on {Γ}_{N} ; \qquad ( 47 )$$

where σ is the Cauchy stress, b is a force, u is the displacement at each node in the domain, Ω is the internal volume domain, Γ_{D} is the set of points on the boundary where we enforce Dirichlet (value-based) boundary conditions, Γ_{N} is the set of points on the boundary where we enforce Neumann (derivative-based) boundary conditions, n is the boundary normal, and Γ_{D} and Γ_{N} have zero intersection and their union is the entire boundary.

Neglecting shear modulus and bulk viscosity, in a viscoelastic tissue model the stress is related to the strain ∈ as:

$$σ = λ tr ( ϵ ) I + 2 η \frac{\partial ϵ}{\partial t} ; \qquad ( 48 )$$

where Lame's first parameter λ is given by:

$$λ = \frac{Ev}{( 1 + v ) {( 1 - 2 v )}^{′}} ; \qquad ( 49 )$$

where E is Young's modulus and v is Poisson's ratio (just less than 1/2), and η is shear viscosity. Assuming that v is a constant just less than 1/2, then inclding the Shear Modulus G given by:

$$G = \frac{E}{2 ( 1 + v )} ; \qquad ( 50 )$$

without increasing the number of variables, modifies the stress equation to:

$$σ = λ tr ( ϵ ) I + 2 G ϵ + 2 η \frac{\partial ϵ}{\partial t} . \qquad ( 51 )$$

However, as can be inferred from (49) and (50), if v is close to ½, then λ>>G due to the 1−2v in λ's denominator, and G may typically be neglected for tissues. Finally, the strain ∈ is related to the displacement u by:

$$ϵ = \frac{1}{2} ( ∇ u + {( ∇ u )}^{T} ) ; \qquad ( 52 )$$

(i.e. strain is the spatial derivative of displacement). The overall equation of motion is given by the Cauchy momentum equation:

$$∇ \cdot σ + b = ρ \frac{{\partial }^{2} u}{\partial {t}^{2}} ; \qquad ( 53 )$$

where ρ is the material density (approximately 1,045 kg/m3 for brain tissue). Substituting (48), another time derivative on u (implicitly, via the time derivative on strain) is obtained:

$$∇ \cdot ( λ tr ( ϵ ) I + 2 η \frac{\partial ϵ}{\partial t} ) + b = ρ \frac{{\partial }^{2} u}{\partial {t}^{2}} . \qquad ( 54 )$$

However, JAX-FEM (or another FEM solver) does or may not solve time-varying problems. Instead, JAX-FEM can be called to solve for the displacement map at each time point in a series.

As such, a finite differencing scheme can be implemented, which marches through time, solving a static problem at each time point i, where time points are separated by a fixed dwell time Δₜ. Denote uᵢ as the displacement field of the current time point, and uᵢ₋₁ and uᵢ₋₂ as the displacement fields at the immediately previous two time points, and so on. Then, finite differencing can be applied to (54) to obtain a new static problem that JAX-FEM or another FEM solver can solve, treating the previous displacement fields as constant terms.

Choices can be made based on desired accuracy. The first-order-accurate formulae for first and second time derivatives are:

$$\frac{\partial u}{\partial t} \approx \frac{1}{{Δ}_{t}} ( {u}_{i} - {u}_{i - 1} ) + O ( {Δ}_{t} ) ; \qquad ( 55 )$$

$$\frac{{\partial }^{2} u}{\partial {t}^{2}} \approx \frac{1}{{Δ}_{t}^{2}} ( {u}_{i} - 2 {u}_{i - 1} + {u}_{i - 2} ) + O ( {Δ}_{t} ) ; \qquad ( 56 )$$

and the second-order-accurate formulae are:

$$\frac{\partial u}{\partial t} \approx \frac{1}{2 {Δ}_{t}} ( 3 {u}_{i} - 4 {u}_{i - 1} + {u}_{i - 2} ) + O ( {Δ}_{t}^{2} ) ; \qquad ( 57 )$$

$$\frac{{\partial }^{2} u}{\partial {t}^{2}} \approx \frac{1}{{Δ}_{t}^{2}} ( 2 {u}_{i} - 5 {u}_{i - 1} + 4 {u}_{i - 2} - {u}_{i - 3} ) + O ( {Δ}_{t}^{2} ) . \qquad ( 58 )$$

Applying the differencing to both the strain and displacement with first-order-accurate differences yields:

$$∇ \cdot ( λ tr ( {ϵ}_{i} ) I + \frac{2 η}{{Δ}_{t}} ( {ϵ}_{i} - {ϵ}_{i - 1} ) ) + {b}_{i} = \frac{ρ}{{Δ}_{t}^{2}} ( {u}_{i} - 2 {u}_{i - 1} + {u}_{i - 2} ) . \qquad ( 59 )$$

λ (via its dependence on E) is in units of Pascal, while η has units of Pascal-seconds, and the seconds is canceled by the time derivative, yielding Pascal. The ∇· operation in (59) introduces a 1/m, so that the overall units of the left term are N/m3 (since Pa is equivalent to N/m²). The body force b is also in N/m³ (as is the convention). The right-hand side of the equation has units kg/(m²-seconds²), because the meters units of u knocks the order of the meters units down by one. At the same time, equivalent units for a Newton are kg-meters/seconds², so division by meters³ yields equivalent left-hand-side units of kg/(m²-seconds²), matching the right-hand-side.

The remaining task then is to determine how the desired acoustic intensity factors into the equation. The time-average acoustic intensity I of an ultrasound beam is expressed in W/m2, the absorption coefficient of the medium is α (Np/m), and c is the speed of sound (m/s). With these quantities defined, the radiation force volume density is given by:

$${Γ}_{a} = \frac{2 α I}{c} ; \qquad ( 60 )$$

which has units N/meters³, matching the units in (59). Hence, bᵢ=Γₐ can be set at each time point when the ultrasound is switched on. The intensity can either b assumed to be uni-axial and coincide with the ultrasound axial dimension, or a three-component vector for the acoustic radiation force can be estimated. This expression relating force and intensity is just one non-limiting example. This example assumes plane waves, which holds well at the focus but degrade with distance. More complicated expressions can be utilized outside of the focus for improved accuracy.

To take this one step further, the acoustic intensity I can be related to the pressure p at a location:

$$I = \frac{{p}^{2}}{Z} ; \qquad ( 61 )$$

$$= \frac{{p}^{2}}{ρ c} ; \qquad ( 62 )$$

where Z is the tissue's acoustic impedance, ρ is its density, and c is the speed of sound.

Then, u_{*}(x, t) can be expressed during the ARF pulse as:

$${u}_{*} ( x , t ) = ( 1 - {e}^{- t / τ} ) {\sum }_{i = 1}^{{N}_{g}} {α}_{i} G ( x , {μ}_{i} , {\sum }_{i} ) ; \qquad ( 63 )$$

where τ is the viscoelastic tissue time constant, N_{g} is the number of Gaussians used to model the u_{*}, aᵢ is the amplitude (bipolar) of Gaussian i, μᵢ is its mean, and Σᵢ is its covariance matrix. After the ARF pulse (t>T where T is the ARF pulse length), the expression becomes:

$${u}_{*} ( x , t ) = ( 1 - {e}^{- T / τ} ) {e}^{- ( t - T ) / τ} {\sum }_{i = 1}^{{N}_{g}} {α}_{i} G ( x , {μ}_{i} , {\sum }_{i} ) ; \qquad ( 64 )$$

where G(x) is given by:

$$G ( x ) = exp ( - \frac{1}{2} {( x - μ )}^{T} {\sum }^{- 1} ( x - μ ) ) . \qquad ( 65 )$$

If we have u(x, t) then we can calculate b using the Cauchy momentum equation as:

$$b ( x ) = - ∇ \cdot σ + ρ \frac{{\partial }^{2} u}{\partial {t}^{2}} . \qquad ( 66 )$$

Calculation of

$$ρ \frac{{\partial }^{2} {u}_{*}}{\partial {t}^{2}}$$

is given, during the pulse, by:

$$ρ \frac{{\partial }^{2} {u}_{*}}{\partial {t}^{2}} = - ρ \frac{1}{{τ}^{2}} {e}^{- t / τ} {\sum }_{i = 1}^{{N}_{g}} {α}_{i} G ( x , {μ}_{i} , {\sum }_{i} ) ; \qquad ( 67 )$$

and, after the pulse, by:

$$ρ \frac{{\partial }^{2} {u}_{*}}{\partial {t}^{2}} = ρ \frac{1}{{τ}^{2}} ( 1 - {e}^{- T / τ} ) {e}^{- ( t - T ) / τ} {\sum }_{i = 1}^{{N}_{g}} {α}_{i} G ( x , {μ}_{i} , {\sum }_{i} ) . \qquad ( 68 )$$

Which shows the utility of the FEM approach.

### EXAMPLE

In one non-limiting example, the above-described systems and methods were studied. First, simulations were performed. Using the process illustrated in FIG. 3A, an 850 kHz single element ultrasound transducer with a 64 mm diameter and 63.2 mm focus was simulated in k-Wave across a range of pressure levels from 0.27 to 2.70 MPa. The average acoustic intensity (I) was calculated from each simulated pressure field and converted to acoustic radiation force using

$$F = \frac{2 α I}{c} ,$$

where I is average pressure-squared, α is the tissue attenuation coefficient, and c is the speed of sound.

The force maps were input to α FEM solver to calculate displacements during MR-ARFI pulses up to 4 ms in duration. FEM-based calculations were performed over a radially symmetric 2.0×2.0×2.5 cm3 slice around the focus with (100,100,150) nodes. The transducer was switched ‘on’ for 4 ms of the 5 ms FEM calculation, corresponding to α motion encoding gradient (MEG) duration up to 4 ms. A Poisson's ratio of 0.49, and a Young's modulus of 2000 Pa were used, the timestep was 5×10−5s. The resulting dynamic displacement maps were used to calculate the expected phase maps for a given motion encoding gradient (MEG) duration, which were then converted back to average displacement during the MEG.

Also, phantom experiments were performed. The focal displacement FWHM's of MR-ARFI maps generated by a Green's theorem PSF-based steady state displacement calculation method and the FEM-simulated ARFI maps were compared to phantom experiments with the transducer described above. Phantom experiments were performed in a 3T Philips Elition MR scanner with an agar-graphite phantom. Because the stiffness of the phantom was unknown, all simulated displacements were normalized to the maximum observed displacement in the phantom, 2.5 μm.

Acoustic pressure fields were simulated across a range of intensities and input to the PSF and FEM methods to calculate MR-ARFI displacements. The minimum, middle, and maximum displacements and corresponding intensities were used as source points in lookup table calculations to interpolate the ‘unknown’ intensities of the middle points, given their displacement. The predicted intensities were compared between PSF- and FEM-based MR-ARFI maps. Though a lookup table was used, artificial intelligence (AI) could also have been used as a tool to carry out the calculations and/or predict intensities.

In these studies, the FWHM of the coronal slices were 10 mm for the PSF method, 2.7 mm for FEM and 6.4 mm for phantom measurements. The axial FWHM were 30.75 mm for the PSF method, 23 mm for FEM and 18.7 mm for phantom measurements. While displacement and intensity varied perfectly linearly with the PSF method, it did not for the FEM method, for either MEG duration. Lookup tables were created to calculate the intensity for a given displacement for the PSF, and FEM methods. The error was less than 3 W/cm² when using FEM source points to interpolate pressure from FEM displacement. When using the PSF method as a source and FEM displacement to calculate the intensity, the error between interpolated intensity and known intensity was 19.25 W/cm², 9% of the true intensity, for FEM with a 0.5 ms MEG, and 29.07 W/cm², 14% of the true intensity for FEM with a 4 ms MEG.

Thus, the studies showed one non-limiting example implementation of systems and methods that provide and can use a comprehensive model of MR-ARFI displacement map generation, which relates acoustic intensity or pressure to displacement, and accounts for time-varying displacement over the duration of a motion encoding gradient/FUS pulse pair. The method was used in an initial study to invert displacement measurements to obtain acoustic intensity. This can then be used in inverse methods for MR-ARFI-based acoustic dosimetry in FUS neuromodulation.

Thus, the present disclosure recognizes that MR-ARFI is currently used to target focused ultrasound in the brain, but as a quantitative imaging method it has the potential to provide acoustic dosimetry if the measured tissue displacement can be related back to acoustic intensity or pressure. The present disclosure provides systems and methods to calculate MR-ARFI images based on high-intensity acoustic simulations of focused ultrasound and finite element modeling. Acoustic beam simulations can be converted to acoustic force, which can be input to an FEM solver to calculate time-resolved tissue displacements, and subsequently MR-ARFI. The systems and methods provided herein provide a lookup table, model, or AI (or other storage or processing resource) to perform calculation to recover acoustic intensity from images. Thus, MR-ARFI can be used to target focused ultrasound in the brain, but as a quantitative imaging method it can also provide dosimetry. The present disclosure established the ability to relate simulated acoustic pressure fields to MR-ARFI images via finite element modeling.

In addition, the present disclosure recognizes that MR-ARFI can be used to solve the problem of determining corrective amplitudes and/or phases (or other mechanisms, such as customized acoustic lenses) to re-focus a focused ultrasound in brain tissue on the other side of the skull, or within the body on the other side of any aberrating medium. Acoustic properties of the aberrating medium can also be determined, such as the attenuation and speed of sound of the skull. In other words, observation of the tissue displacement in MR-ARFI can be used to tomographically solve for the properties of the skull that the sound passed through to reach the displaced tissue. To date however, only data-driven methods have been described to achieve this goal, in which adjustments are iteratively made to the individual elements of a multi-element transducer to maximize tissue displacement in a target region in the brain or body, or a large number of MR-ARFI images are collected and combined to determine complex-valued continuous-wave pressure maps for individual transducer elements or groups of elements.

The present disclosure recognizes that one can create systems and methods to fully model the process of generating an MR-ARFI image, compare a modeled image to α measured image, then backpropagate derivatives with respect to any system parameter to estimate that parameter. As will be described, this can be done, for example, using auto-differentiation methods for both acoustic simulation (e.g. j-Wave, such as described in Stanziola, Antonio, et al. “j-Wave: An open-source differentiable wave simulator.” SoftwareX 22 (2023): 101338., which is incorporated herein by reference) and/or finite element modeling (e.g. Jax-FEM, such as described in Xue, Tianju, et al. “JAX-FEM: A differentiable GPU-accelerated 3D finite element solver for automatic inverse design and mechanistic data science.” Computer Physics Communications (2023): 108802., which is incorporated herein by reference).

In particular, the above-described systems and methods for using a differentiable FEM solver to calculate the tissue displacement produced by a FUS push, can be used to simultaneously determine acoustic intensity and tissue elasticity or other properties. In some situations, the above-described systems and methods may be adapted to simultaneously fit to more than one MR-ARFI image collected with different sequence parameters. For example, to fit to multiple parameters, multiple delays may be used between the FUS pulses and the motion encoding gradients of the MR-ARFI sequence.

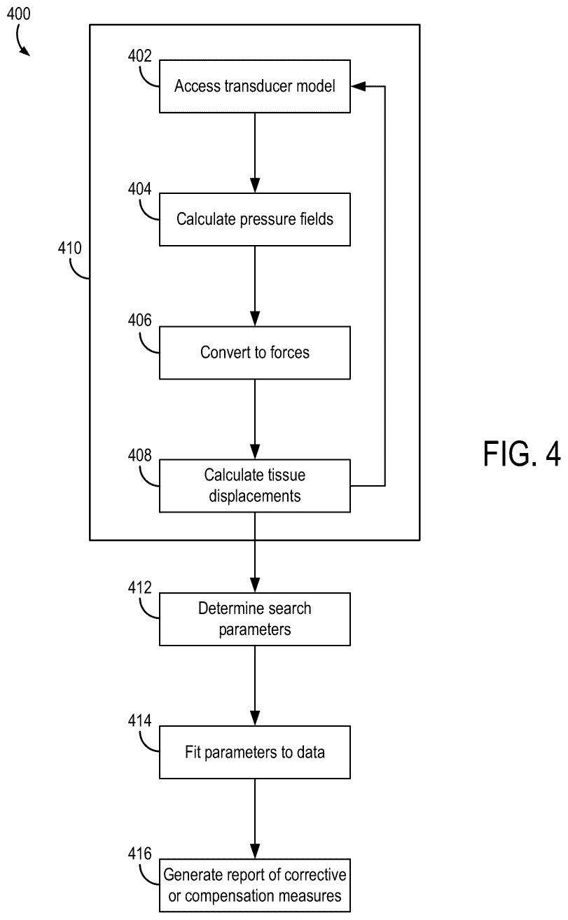

Referring to FIG. 4, steps of a non-limiting example method 400 for estimating tissue acoustic (rather than or in addition to mechanical or elastic) parameters are provided. the process 400 begins at process block 402 with accessing a model of a FUS or TUS transducer and a tissue medium. The process 400 continues at process block 404 by calculating the pressure fields generated by the transducer in the tissue medium using a differentiable acoustic solver, such as j-Wave or JAX-FEM. At process block 406, those pressures are converted to forces and, at process block 408, tissue displacements are calculated using a finite element method solver which may or may not be differentiable, and using the tissue displacements to calculate an MR-ARFI image.

The above described steps form a functional block 410 that yields tissue displacements. Functional block 410 can be repeated with different parameters, such as delays between motion encoding gradients and the FUS pulse, and one or more collected MR-ARFI images.

At process block 412, a search direction to update same parameters may be determined. For example, the derivatives can be backpropagated with respect to parameters of the transducer or tissue medium back from the inputs to the FEM solver to determine a search direction to update the parameters of interest. In some non-limiting examples, the of interest parameters may be acoustic velocities or attenuations of the tissue or bone, or effective amplitudes and phases of individual elements of the ultrasound transducer.

Using the derivatives determined at process block 412, at process block 414, the parameters of interest may be fit to the observed MR-ARFI data. For example, an iterative algorithm or AI may be used to select the parameters that fit the observed MR-ARFI data. At process block 416, using the determined parameters of interest, a report of corrective or compensation measures can be generated and/or reported. As one example, the corrective or compensation measures can include the driving amplitudes and phases or other corrective measures. For example, an acoustic lens that refocus the ultrasound beam to an intended target can be selected that overcomes the aberrations or imperfections that were identified at process block 414. As a further example, if a set of effective driving amplitudes and phases are determined at process block 414, applying a pulse with the inverses and conjugates of those amplitudes and phases (respectively) will result in a focused beam at the intended target.

Thus, the present disclosure recognizes that MR-ARFI can be used to solve the problem of determining corrective amplitudes and/or phases (or other mechanisms, such as customized acoustic lenses) to re-focus a focused or transcranial ultrasound process. Acoustic properties of the aberrating medium can be determined, such as the attenuation and speed of sound of the skull or abnormality.

It is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The preceding discussion was presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

In some configurations, any suitable computer-readable media can be used for storing instructions for performing the functions and/or processes described herein. For example, in some configurations, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present disclosure has described one or more preferred embodiments, and it should be appreciated that many equivalents, alternatives, variations, and modifications, aside from those expressly stated, are possible and within the scope of the invention.

1. A method to create a magnetic resonance acoustic radiation force imaging (MR-ARFI) image from simulations or measurements of a pressure field of an ultrasound transducer, the method comprising:
converting simulations or measurements of a pressure field to force;
delivering the force to a model to calculate dynamic tissue displacements in tissue; and
delivering the dynamic tissue displacement to control operation of an MR-ARFI process.

2. The method of claim 1, further comprising recovering a characteristic of the pressure field from MR-ARFI-measured displacement maps.

3. The method of claim 2, further comprising using a mathematical relationship between the pressure field and the MR-ARFI-measured displacement map stored in a neural network or lookup table.

4. The method of claim 2, wherein the model is configured to assume the tissue has mechanical properties that are uniform or are measured using shear wave elastography or MR elastography.

5. The method of claim 1, further comprising:
accessing a model of a focused ultrasound transducer and a tissue medium;
calculating the pressure field generated by the ultrasound transducer in a tissue medium of a patient;
calculating tissue displacements in the patient using the pressure fields using a model;
generating an MR-ARFI image using the tissue displacements and MR-ARFI image data acquired from the patient during the MR-ARFI process;
using the MR-ARFI image, backpropagate derivatives with respect to parameters of the ultrasound transducer or the tissue medium back from the inputs to the model;
using the derivatives, selecting updated parameters of the ultrasound transducer or the tissue medium that fit the MR-ARFI image data; and
using the updated parameters, select corrective measures that refocuses an ultrasound beam from the ultrasound transducer to an intended target through aberrations or imperfections in the tissue medium.

6. A non-transitory computer storage medium having instructions stored thereon that, when executed by a processor, cause the processor to carry out steps comprising:
accessing parameters of ultrasound transducer configured to generate a range of pressure levels;
calculating displacements during MR-ARFI pulses based on the generated pressure levels; and
determining unknown intensities based on the displacements.

7. The storage medium of claim 6, wherein the ultrasound transducer is a single element ultrasound transducer configured to generate pressure levels ranging from 0.27 to 2.70 MPa.

8. The storage medium of claim 6, wherein the processor is configured to use a finite element method (FEM) to calculate the displacements during MR-ARFI pulses.

9. The storage medium of claim 8, wherein the processor is configured to use the FEM to perform calculations over a radially symmetric slice around focus nodes.

10. The storage medium of claim 9, wherein the processor is configured to use the FEM with a Poisson's ratio of 0.49, and a Young's modulus of 2000 Pa, and a timestep of 5×10−5s.

11. The storage medium of claim 6, wherein the processor is configured to access a lookup table to determine the unknown intensities.

12. The storage medium of claim 11, wherein the lookup table includes uses minimum, middle, or maximum displacements and corresponding intensities as source points in calculations to interpolate the unknown intensities.

13. The storage medium of claim 12, wherein the unknown intensities are determined at middle points.

14. A method comprising:
accessing a range of pressure levels generated or capable of being generated in a subject using an ultrasound transducer during a transcranial ultrasound process (TUS) or a focused ultrasound process (FUS);
using the computer processor, processing the pressure levels with a model that calculates displacements during a magnetic resonance acoustic radiation force imaging (MR-ARFI) process;
using the computer processor, interpolating unknown intensities using the displacements; and
using the computer processor, communicating the displacement to α system performing one of a the TUS or FUS process to facilitate control of the TUS process or FUS process.

15. A system for estimating tissue mechanical or acoustic properties of a subject using magnetic resonance acoustic radiation force images (MR-ARFI), the system comprising:
a processor configured to:
access a model of a focused ultrasound transducer and a tissue medium;
control the focused ultrasound transducer to acquire MR-ARFI image data from an intended target through the aberrations or imperfections;
calculate pressure fields generated by the focused ultrasound transducer in a tissue medium of the subject;
calculate tissue displacements using the pressure fields using a model;
generate an MR-ARFI image using the tissue displacements and MR-ARFI image data acquired from the subject;
using the MR-ARFI image, backpropagate derivatives with respect to parameters of the focused ultrasound transducer or the tissue medium back from the inputs to the model;
using the derivatives, select updated parameters of the focused ultrasound transducer or the tissue medium that fit the MR-ARFI image data; and
using the updated parameters, select corrective measures that refocuses an ultrasound beam from the focused ultrasound transducer to the intended target through the aberrations or imperfections.

16. The system of claim 15, wherein the processor is further configured to calculate the pressure fields by simulating the propagation of ultrasound waves from the focused ultrasound transducer through the tissue medium.

17. The system of claim 16, wherein the processor is further configured to convert the pressure fields to forces.

18. The system of claim 17, wherein the processor to configured to calculate tissue displacements by simulating a response of the tissue medium to the forces calculated.

19. The system of claim 18, wherein the processor is configured to calculate the MR-ARFI image by encoding the tissue displacements into the MR-ARFI image to create an updated MR-ARFI image.

20. The system of claim 19, wherein the processor is further configured to adjust parameters of the focused ultrasound transducer or tissue medium based on a comparison between the updated MR-ARFI image and the MR-ARFI image.

21. A method for estimating tissue mechanical or acoustic properties of a subject using magnetic resonance acoustic radiation force images (MR-ARFI), the method comprising:
accessing a model of a focused ultrasound transducer or a tissue medium;
controlling the focused ultrasound transducer to acquire MR-ARFI image data from an intended target through the aberrations or imperfections;
using a differentiable acoustic solver, calculating pressure fields generated by the focused ultrasound transducer in a tissue medium of the subject;
using a finite element method (FEM) solver, calculating tissue displacements using the pressure fields;
generating an MR-ARFI image using the tissue displacements and MR-ARFI image data acquired from the subject;
using the MR-ARFI image, backpropagating derivatives with respect to parameters of the focused ultrasound transducer or the tissue medium back from the inputs to the FEM solver;
using the derivatives, selecting updated parameters of the focused ultrasound transducer or the tissue medium that fit the MR-ARFI image data; and
using the updated parameters, selecting corrective measures that refocuses an ultrasound beam from the focused ultrasound transducer to the intended target through the aberrations or imperfections.
